(19) United States
(12) Patent Application Publication
(10) Pub. No.: US 2007/0244362 A1
(43) Pub. Date: Oct. 18, 2007

(54) SEGMENTED COLONOSCOPE

(76) Inventor: Roger El-Hachem, Mansfield, CT (US)

Correspondence Address: MCCORMICK, PAULDING & HUBER LLP, CITY PLACE II, 185 ASYLUM STREET, HARTFORD, CT, 06103, US

(21) Appl. No.: 11/734,391

(22) Filed: Apr. 12, 2007

Related U.S. Application Data
(60) Provisional application No. 60/791,515, filed on Apr. 12, 2006.

(51) Int. Cl. A61B 1/00

(52) U.S. Cl. 600/144; 600/141; 600/146; 600/128

(57) ABSTRACT

A colonoscope includes an elongated body extending between a proximal and a distal end, a tip arranged on the distal end of the elongated body, and a handle arranged on the proximal end of the elongated body, wherein the elongated body generally increases in diameter and flexibility from the distal end to the proximal end. The elongated body preferably has a plurality of segments, each more proximal segment having a larger diameter than each adjacent more distal segment and/or each more proximal segment being more flexible than each adjacent more distal segment. A method for manufacturing a colonoscope includes selecting a length for each segment such that a most distal segment will extend far enough into a corresponding most distal section of the colon to be examined with each more proximal segment advanced through a corresponding more proximal section of the colon.

### CROSS-REFERENCE TO RELATED APPLICATIONS

This application claims the benefit of U.S. Provisional Application No. 60/791,515, filed on Apr. 12, 2006, the contents of which are herein incorporated by reference in their entirety.

### FIELD OF THE INVENTION

The present invention relates to endoscopes, and more particularly relates to colonoscopes employed in the performance of colonoscopies.

### BACKGROUND OF THE INVENTION

A colonoscopy is a medical procedure in which the entire length of the colon is examined. Typically, a colonoscopy is performed to look for tumors or polyps, or to identify the source of gastrointestinal bleeding, although other diagnostic and therapeutic modalities are also known.

A colonoscope is a special type of endoscope used to perform colonoscopies. Colonoscopes are instruments with elongated, flexible bodies. A distal end of the colonoscope is inserted through the rectum and guided through the length of the colon. The distal end includes a distal tip containing a small camera, a light source, as well as biopsy and irrigation channels.

The camera, light source, and biopsy and irrigation channels are connected to corresponding support equipment in a separate equipment tower. The support equipment includes a video imaging system, a light source, and suction and irrigation mechanisms. The camera, light source, and biopsy and irrigation channels are connected to the corresponding support equipment by means of connections running through the interior of the elongated, flexible body of the colonoscope.

Opposite the distal end of the elongated, flexible body is a proximal end including a handle for use in manipulating the colonoscope. The handle has a knob for steering the distal tip, typically by means of wires running through the interior of the elongated, flexible body. The support equipment connections, which also run through the interior of the elongated, flexible body, exit the colonoscope at the proximal end.

During a colonoscopy, the colonoscope is inserted into the colon through the rectum and navigated, in order, through the sigmoid colon, the descending colon, the transverse colon and the ascending colon. Once the ascending colon has been navigated, inspection of the cecum typically completes the procedure. The cecum connects the colon with the ileum, or the final section of the small intestine.

Because of the curvature and redundancy of the various sections of the colon, and because of friction between the colonoscope and the colonic wall, difficulties are frequently encountered during the performance of the colonoscopy, such as coiling of the colonoscope within the colon. If the colonoscope coils, at least a partial withdrawal is usually necessary prior to continuing the colonoscopy.

Various pushing and twisting maneuvers are used to facilitate the advancement of the colonoscope, as is physical manipulation of the patient, such as pushing of the patient's abdomen and turning the patient from side to side. However, current colonoscopes are still difficult and time-consuming to insert, result in substantial pain and discomfort for the patient, and risk more serious complications such as perforation of the colonic wall, and other colon damage.

In view of the foregoing, it can be seen that there is a need for a colonoscope that is more resistant to coiling, more quickly and easily navigated through the entire length of the colon, with less pain and discomfort to the patient, and lower risk of serious complications.

### SUMMARY OF THE INVENTION

According to an embodiment of the present invention, a colonoscope includes an elongated body extending between a proximal and a distal end, a tip arranged on the distal end of the elongated body, and a handle arranged on the proximal end of the elongated body, wherein the elongated body generally increases in diameter and flexibility from the distal end to the proximal end.

According to an aspect of the present invention, the elongated body has a plurality of segments, each more proximal segment having a larger diameter than each adjacent more distal segment and/or each more proximal segment being more flexible than each adjacent more distal segment.

According to another embodiment of the present invention, a method for manufacturing a colonoscope includes the steps of selecting a number of segments for the colonoscope corresponding to the number of sections of a colon to be examined during a complete colonoscopy, selecting a length for each segment such that a most distal segment will extend far enough into a corresponding most distal section of the colon to be examined with each more proximal segment advanced through a corresponding more proximal section of the colon, and assembling a colonoscope based on the selections.

Advantageously, a colonoscope according to embodiments and aspects of the present invention, allows for quicker and easier intubation of the colon, minimizing resistance between the colonoscope and the colon, reducing the chances of coiling, and decreasing pain, discomfort and the risk of serious complications.

These and other advantages, objects, and features of the present invention will be better understood in view of the drawings and detailed description.

### BRIEF DESCRIPTION OF THE DRAWINGS

FIG. 1 is a schematic view of a colonoscope, according to an embodiment of the present invention.

### DETAILED DESCRIPTION OF A PREFERRED EMBODIMENT

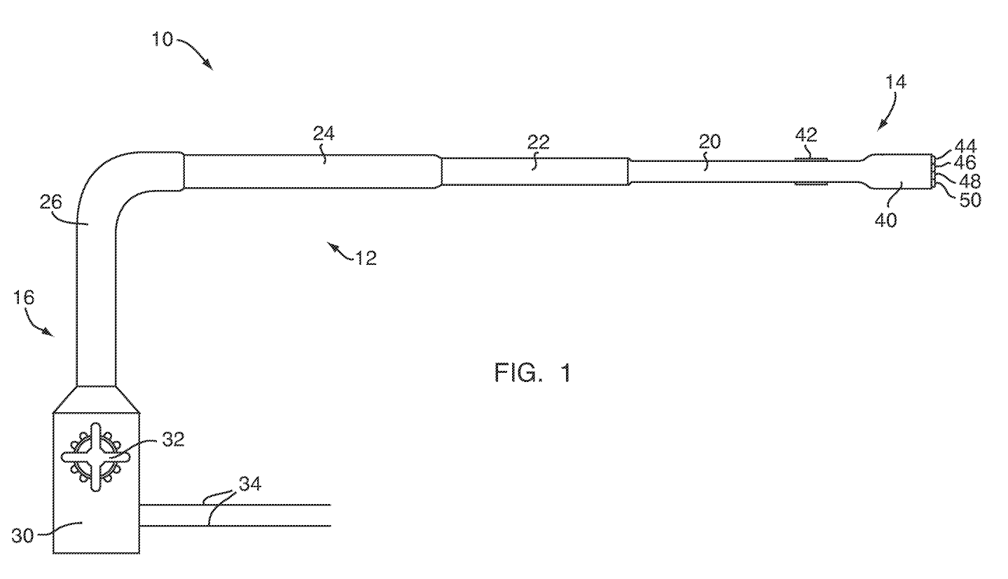

Referring to FIG. 1, a colonoscope 10 includes an elongated body 12, extending between a distal end 14 and a proximal end 16. The elongated body 12 is formed from a plurality of segments, including a first segment 20, a second segment 22, a third segment 24, and a fourth segment 26.

The proximal end 16 includes a handle 30, having a knob 32 located thereon. A plurality of connections 34 to an equipment tower (not shown) exit the proximal end 16. The distal end 14 includes a distal tip 40. The distal tip 40 is connected to the first segment 20 of the elongated body 12 by a joint 42. Using the knob 32, the distal tip 40 can be turned relative to the first segment 20 in various directions.

Located within the distal tip 40 are an irrigation channel 44, a light source 46, a camera 48 and an suction channel 50. The irrigation channel 44, the light source 46, the camera 48, and the suction channel 50 are connected to the equipment tower by the connections 34, which run through the elongated body. The distal tip 40 is approximately 4 cm in length and 10 mm in diameter, and is contoured to smoothly slide against the inner surface of the colonic wall, thus preventing damage.

Control wires (not shown) running between the knob 32 and the distal tip 40 are used to allow manipulation of the distal tip 40 by a user of the colonoscope 10. U.S. Pat. No. 5,989,182, the contents of which are herein incorporated by reference in their entirety, discloses an example of a endoscopic steering device suitable for use with the colonoscope 10.

The first segment 20 is the least flexible of the four segments, and the narrowest in diameter. The first segment 20 is preferably approximately 25 cm in length and 5 mm in diameter. The relative rigidity of the first segment 20 inhibits coiling, while the small diameter results in lower friction with the colonic wall, particularly when navigating the sigmoid and descending colon. Ultimately, the first segment 20 is navigated all the way through the ascending colon to the cecum, permitting inspection of the cecum.

The second segment 22 is more proximal than the adjacent, more distal, first segment 20, and is both more flexible and wider in diameter than the first segment 20. Preferably, the second segment 22 is approximately 35 cm in length and 7 mm in diameter. The diameter of the second segment 22 is still small enough to minimize friction, while large enough to offset the tendency to coil created by the still relatively high flexibility. To advance the first segment 20 into the ascending colon to the cecum, it will typically only be necessary to navigate the second segment 22 up to and through the transverse colon.

The third segment 24 is more proximal than the adjacent, more distal, second segment 22, and is both more flexible and wider in diameter than the second segment 22. Preferably, the third segment 24 is approximately 35 cm in length and 9 mm in diameter. The relatively greater friction created by the larger diameter of the third segment 24 is not substantially deleterious to insertion because the third segment 24 is not required to navigate as far into the colon as the first and second segments 20, 22. Usually, the third segment will be navigated up to and through the descending colon.

The fourth segment 26 is the most proximal segment, and is both the most flexible and the widest in diameter. Preferably, the fourth segment 26 is approximately 35 cm in length and 11 mm in diameter. The fourth segment 26 will generally only be required to navigate the rectum and the sigmoid colon, and support the first, second and third segments 20-24.

To reduce the flexibility of the narrower distal segments various stiffening means can be employed, such as stiffening inserts placed in the interior of the segments, increased wall thickness in the stiffer segments, stiffening structures, for instance longitudinal ridges, formed on the interior of the segments, and the like. Also, a segment can be formed from a number of smaller jointed portions. The desired flexibility of a given segment is then achieved by varying the number of portions and/or changing the range of motion of the joints between the portions. For example, to make a segment more flexible the number of portions per unit length could be increased and/or the range of motion of the joints between the portion could be increased.

It is further preferred that transitions between adjacent segments be made smoothly, to avoid sharp or protruding surfaces that might damage the colonic wall. Accordingly, the plurality of segments of the elongated body 12 can be commonly covered in a smooth outer sheath.

In use, the colonoscope 10 is inserted and navigated through the colon in the same manner as current colonoscopes. However, it will be appreciated that the colonoscope 10, as in the preferred embodiment, having the elongated body 12, with a flexibility and a diameter which both increase from the distal end 14 to the proximal end 16, permits the relatively narrow and rigid distal end 14 to be more quickly navigated through the colon with lower friction and risk of coiling, while being adequately supported by a more flexible yet wider proximal end 16.

It will be further appreciated that forming the elongated body 12 from a plurality of segments offers a practical way for achieving the increase in flexibility and diameter of the elongated body from the distal to the proximal end. The segmented body 12 is further advantageous when the quantity of segments is chosen to substantially correspond to the sections of the colon, as in the described embodiment, where the lengths of the first, second, third and fourth segments 20-26 substantially corresponds to each segment's ultimate advancement through the ascending, transverse, descending, and sigmoid colons, respectively.

The present invention is not limited to the embodiment herein described. Instead, those skilled in the art will recognize that various modifications and adaptations to circumstances fall within the scope of the present invention.

For example, the colonoscope 10 is described having an elongated body 12 formed from four discrete segments, with preferred dimensions. No two colons are exactly alike, and dimensions of the preferred embodiment are selected, in part, based on the average structure and dimensions of an adult colon. The number and dimensions of segments can be varied, as appropriate.

Also, the flexibility and/or diameter of the elongated body varied continuously for all or a portion of the length of the elongated body. Accordingly, a general change in diameter and flexibility over the length of the elongated body can be either a change in stepped increments between segments, a continuous change not necessarily corresponding to discrete segments, or a combination of both. For instance, the diameter could change in stepped increments between segments while the flexibility changes continuously. Also, one or more discrete segments could be tapered, such that there was a change in diameter not only between segments, but also within a given segment.

Additionally, the term “diameter” does not necessarily imply an elongated body or segment thereof with a circular cross-sectional, and should be understood to encompass equivalent dimensions for elongated bodies having other cross-sectional profiles.

Colonoscopes made from various materials all fall within the scope of the present invention. However, suitable bio-compatible materials, as are known in the art, are most appropriate for construction of the colonoscope 10. Polymers, such as polyurethane, polyester, polyethylene, silicone, and the like are examples of suitable materials for the outer sheath and/or segments, though other materials or combinations of materials can be used, to suit particular circumstances. Bio-compatible metals, such as stainless steel, can also be used for some colonoscope components.

It should also be appreciated that a colonoscope according to the present invention, having a flexibility and a diameter increasing from a distal to a proximal end, can still have local discontinuities in flexibility and diameter, for example at the junctions of the segments, while still having a general change in flexibility and diameter falling within the scope of the present invention.

Also, the present invention is not limited to a particular flexibility for a given segment, unless otherwise indicated, so long as the relative flexibility between the segments is preserved. In one preferred embodiment, the flexibility of the most proximal segment is approximately the same flexibility as is found in currently-available colonoscopes.

Additionally, it will be appreciated that other control mechanisms, besides the knob 32, and devices other than irrigation channel 44, light source 46, camera 48, and suction channel 50, can be advantageously incorporated into the present invention, without departing from the scope thereof. Also, various materials and stiffening means can be used within the scope of the present invention.

The preceding variations are not an exclusive list, but are merely examples of the type of variations, modifications, and adaptations falling within the scope of the present invention.

What is claimed is:

1. A colonoscope comprising:
an elongated body extending between a proximal and a distal end;
a tip arranged on the distal end of the elongated body; and
a handle arranged on the proximal end of the elongated body;
wherein the elongated body generally increases in diameter and flexibility from the distal end to the proximal end.

2. The colonoscope of claim 1, wherein the elongated body has a plurality of segments, each more proximal segment having a larger diameter than each adjacent more distal segment.

3. The colonoscope of claim 1, wherein the elongated body has a plurality of segments, each more proximal segment being more flexible than each adjacent more distal segment.

4. The colonoscope of claim 3, wherein each more proximal segment has a larger diameter than each adjacent more distal segment.

5. The colonoscope of claim 4, wherein the elongated body has four segments, the four segments being, from the distal to the proximal end, a first, second, third and fourth segment.

6. The colonoscope of claim 5, wherein each segment has a predetermined length, the length being predetermined to substantially correspond to each segment's ultimate advancement through average ascending, transverse, descending, and sigmoid colons, respectively, during a colonoscopy.

7. The colonoscope of claim 6, wherein the length of the first segment is approximately 25 cm, and the length of each of the second, third and fourth segments is approximately 35 cm.

8. The colonoscope of claim 7, wherein the diameters of the first, second, third and fourth segments are, respectively, approximately 5 mm, approximately 7 mm, approximately 9 mm and approximately 11 mm.

9. The colonoscope of claim 1, wherein the colonoscope further comprises a sheath of biocompatible material substantially encasing the elongated body.

10. The colonoscope of claim 1, wherein the tip includes at least one of an irrigation channel, a light source, a camera, and a suction channel.

11. The colonoscope of claim 10, wherein the at least one of the irrigation channel, light source, camera and suction channel has a corresponding connection running through the elongated body and exiting the handle.

12. The colonoscope of claim 1, wherein the tip is connected to the distal end of the elongated body by a joint allowing the tip to be turned relative to the distal end.

13. The colonoscope of claim 12, wherein the handle includes a knob connected to the tip by at least one wire, such that operating the knob turns the tip relative to the distal end.

14. A method for manufacturing a colonoscope, the method comprising the steps of:
selecting a number of segments for the colonoscope corresponding to the number of sections of a colon to be examined during a complete colonoscopy;
selecting a length for each segment such that a most distal segment will extend far enough into a corresponding most distal section of the colon to be examined with each more proximal segment advanced through a corresponding more proximal section of the colon; and
assembling a colonoscope based on the selections.

15. The method of claim 14, further comprising the step of selecting a flexibility for each segment such that each more distal segment is less flexible than each adjacent more proximal segment.

16. The method of claim 14, further comprising the step of selecting a diameter for each segment such that each more distal segment is narrower than each adjacent more proximal segment.

17. A colonoscope comprising:
an elongated body extending between a distal and a proximal end, and having a plurality of segments each more distal segment being less flexible and narrower than each adjacent more proximal segment;
a tip arranged on the distal end of the elongated body; and
a handle arranged on the proximal end of the elongated body.

18. The colonoscope of claim 17, wherein the elongated body consists of four segments.

19. The colonoscope of claim 18, wherein lengths of each segment are such that, with the colonoscope fully inserted through an average rectum, sigmoid colon, descending colon, transverse colon and ascending colon into a cecum, a most proximal, of the four segments is long enough to extend through the rectum and the sigmoid colon, a second-most proximal, of the four segments is long enough to extend through the descending colon, a third-most proximal of the four segments is long enough to extend through the transverse colon, and a most distal of the four segments is at least long enough to extend through the ascending colon into the cecum.
